(19) United States
(12) Patent Application Publication
(10) Pub. No.: US 2005/0165482 A1
(43) Pub. Date: Jul. 28, 2005

(54) Bone fixation element

(76) Inventors: Jorg Goldhahn, Dubendorf (CH); Jorn Seebeck, Wiuterthur (CH); Erich Schneider, Davos (CH)

Correspondence Address: JONES DAY, 222 EAST 41ST STREET, NEW YORK, NY, 10017-6702, US

(21) Appl. No.: 11/021,999

(22) Filed: Dec. 22, 2004

Related U.S. Application Data
(63) Continuation of application No. PCT/CH02/00347, filed on Jun. 26, 2002.

(51) Int. Cl. A61B 17/56

(52) U.S. Cl. 623/16.11

(57) ABSTRACT

A bone fixation element consisting of a hollow member having a wall, a plurality of perforations locating in the wall, a front end which is suited for insertion into the bone, a rear end, and a longitudinal axis. The stiffness of the hollow member may vary along its longitudinal axis from the rear end to the front end. Changes in the porosity and/or the thickness of the wall of the bone fixation element may result in changes in stiffness of the bone fixation element. An increase in porosity and/or decrease in thickness may result in a decrease in stiffness, and a decrease in porosity and/or increase in thickness may result in an increase in stiffness.

### CROSS REFERENCE TO RELATED APPLICATION

The present application is a continuation of International Patent Application PCT/CH2002/000347 filed Jun. 26, 2002, the entire content of which is expressly incorporated herein by reference thereto.

### FIELD OF THE INVENTION

The invention relates to a bone fixation element and, in particular, to a device for insertion into bone that promotes bone growth and has enhanced bone anchoring capability.

### BACKGROUND OF THE INVENTION

Existing implants have inadequately addressed the reactions of the surrounding tissue, especially of the bone, to a foreign body, namely the implant. In particular, the effect of using relatively stiff implants, such as those made of titanium or steel, has inadequately been taken into consideration. When implants are used, the local microcirculation or perfusion is damaged and the local biomechanics are changed.

In order to prevent fatigue breakages, implants have been designed to be as stiff and as stable as possible. While a bone fracture is healing and during the entire time that the implant remains in the body, the implant at least partly assumes the load transfer function of the bone. As a result, there may be a thinning of the bone structure in the regions around the bone implant (i.e., the bones adapt according to Wolff's Law). Thinning of the bone, in turn, may lead to a loosening of the implant.

In particular, hollow cylinders, such as those provided for the medical care of fractures in osteoporotic bones and disclosed in Patent Nos. DE-C 19628473 and DE-U 297 10 979, can lead to thinning. However, since one would expect hollow cylinders to fail less frequently, the use of hollow cylinders may be advantageous for problems caused by osteoporosis. The bone in the interior of a hollow cylinder should be retained, and the new bone should grow through perforations formed in the hollow cylinder. Theoretically, this should lead to a better anchoring of the implant in osteoporotic bone.

Damage to the microcirculation and perfusion may result from the supplying blood vessels being severed during the insertion of an implant. Investigations have shown that a recovery of the blood supply inside and outside of the hollow cylinder is possible within a very short time. Perforations in the wall of the hollow cylinder as well as cyclic compressions, which result from a perfusion of tissue fluid, are important for supplying blood to the interior of the hollow cylinder. The extent of the perfusion of tissue fluid, in turn, depends on the stiffness of the implant. Moreover, cyclic compression produced by the application of a load on the bone during the healing process also leads to the regeneration of adjacent bone sections. Therefore, the bony integration of an implant depends on the implant's mechanical properties, especially on the implant's stiffness.

### SUMMARY OF THE INVENTION

The present invention provides a bone fixation element which, due to its construction, permits the stiffness of the bone fixation element to be varied and, thus, may ensure better long-term anchorage in the bone, especially in the osteoporotic bone. At the same time, the bone fixation element may provide benefits to the area around the implant, may promote the growth of new bone, and assures the retention of remaining bony structures.

In one embodiment, the bone fixation element may be a hollow member, preferably a hollow cylinder, having a wall with a plurality of perforations. The wall may be a circumferential surface and the member may have an opened front end for inserting into bone. The member may also have an externally threaded portion to engage the surrounding bone or tissue. In addition, the rear end of the hollow member may be provided with means (e.g., a conical portion) for accommodating, for example, a bone plate.

The stiffness of the bone fixation element may vary along the length of the bone fixation element. In one embodiment, the stiffness of the bone fixation element may decrease from the rear end to the front end of the bone fixation element. The stiffness of the bone fixation element may be a function of the porosity and/or the thickness of the wall of the bone fixation element. An increase in porosity and/or a decrease in thickness of the wall may result in a decrease in stiffness. Similarly, a decrease in porosity and/or an increase in thickness of the wall may result in an increase in stiffness. In some embodiments, the porosity of the bone fixation element may increase continuously and/or the thickness of the wall of the bone fixation element may decrease continuously from the rear end to the front end of the bone fixation element.

The porosity may be dependent on the number of perforations and/or the size of the perforations in the bone fixation element. The porosity of the bone fixation element may be increased by increasing the size of the perforations and/or the number of perforations. Similarly, the porosity of the bone fixation element may be decreased by decreasing the size of the perforations and/or the number of perforations. Moreover, the porosity of the bone fixation element may be modified by changing the shape of the perforations.

In one embodiment, the hollow member may have a first stiffness at the front end and a second stiffness at the rear end, a first thickness at the front end and a second thickness at the rear end, and a first porosity at the front end and a second porosity at the rear end. The first stiffness may be less than the second stiffness, the first thickness may be less than the second thickness and the first porosity may be greater than the second porosity.

### BRIEF DESCRIPTION OF THE DRAWINGS

The invention and further developments of the invention are explained in even greater detail in the following exemplary drawings. The present invention can be better understood by reference to the following drawings, wherein like references numerals represent like elements. The drawings are merely exemplary to illustrate certain features that may be used singularly or in combination with other features and the present invention should not be limited to the embodiments shown.

FIG. 1 is a perspective view of an exemplary embodiment of the present invention which is partly cut open;

FIG. 2 is a partial view of an exemplary embodiment of the wall of the device of the present invention with a number of cross sections through the wall;

FIG. 3 is a partial view of an alternative exemplary embodiment of the wall of the device of the present invention with oval perforations;

FIG. 4 is a partial view of an alternative exemplary embodiment of the wall of the device of the present invention with square perforations; and

FIG. 5 is a partial view of an alternative exemplary embodiment of the wall of the device of the present invention with triangular perforations.

### DETAILED DESCRIPTION

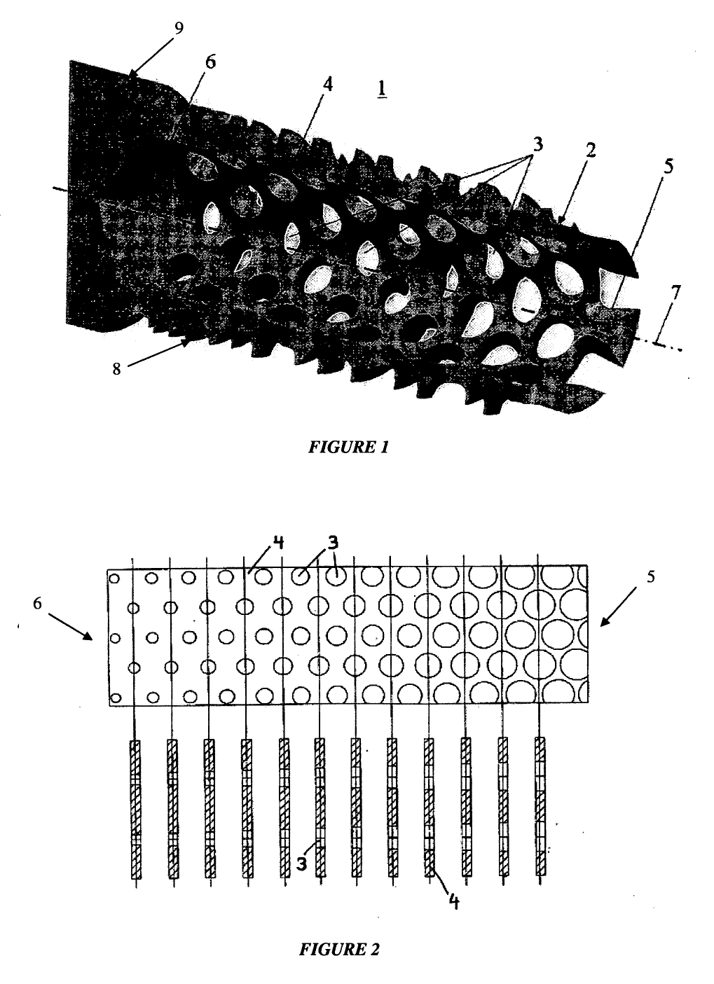

As shown in FIG. 1, the bone fixation element 1 may be in the form of a hollow member 2 having a wall 4, a plurality of perforations 3 in the wall 4, a front end 5 suitable for introduction into bone, a rear end 6 and a longitudinal axis 7. Preferably, the hollow member 2 may be in the form of a cylinder with an opened front end 5. Moreover, the wall 4 may be a circumferential surface. It should, however, be understood that those of ordinary skill in the art will recognize many modifications and substitutions which may be made to various elements of the present invention.

As shown, at least a portion of the wall 4 may be provided with an external thread 8 for engaging bone or tissue. The thread 8 may be on a portion adjacent the rear end 6. Additionally, the rear end 6 of the bone fixation element 1 may be provided with means, for example, an enlarged portion or conical head 9, which may be inserted in/attached to a bone plate (not shown).

Some of the advantages achieved by the bone fixation element 1 of the present invention reside in the fact that the stiffness of the hollow member 2 may be approximated to that of the surrounding bone. For example, the stiffness of the bone fixation element 1 may conform to the different stiffness present in a vertebrae between the corticalis, which has a modulus of elasticity of approximately 10,000 to 20,000 MPa, and the spongiosa, which has a modulus of elasticity of approximately 100 to 5,000 MPa. In addition, the bone fixation element 1 may have varying stiffness along its length to conform to conditions where the deflection and compression of the bone fixation element 1 under load is greater in the interior of the vertebrae than in the circumference or edge region of the vertebrae.

The stiffness of the bone fixation element 1 may change along the longitudinal axis 7 by, for example, (1) reducing the thickness of the wall 4 of the bone fixation element 1 in the region of transition between different portions of bone (e.g., between the corticalis and the spongiosa) and/or (2) increasing the porosity in the direction of the front end 5 of the hollow member 2. A reduction in wall thickness and/or an increase in porosity may result in a decrease in stiffness. Conversely, an increase in wall thickness and/or a reduction in porosity may result in an increase in stiffness.

In the case of a thin-walled bone fixation element 1 with a circular profile of average radius Rₘ, the axial and polar areal moments of inertia (Iₓ and Iₚ), as measurements of the stiffness, may depend roughly on the wall thickness t and the porosity p (perforation area/total surface area) in the following way:
Iₓ≈Π·Rₘ³·t·p
Iₚ≈2·Π·Rₘ³·t·p

In one embodiment, the stiffness of the bone fixation element 1 may decrease continuously from the rear end 6 to the front end 5. Moreover, the stiffness of the rear third of the hollow member 2 may be, for example, at least about 20% greater than the stiffness of the front third of the hollow member 2.

By parametric finite element analysis, it was possible to establish that the stiffness of a circular section of the bone fixation element 1 depends exponentially on the wall thickness.
W_{cross section}˜t^{2.7343} is valid when t/Rₘ={0.4 . . . 0.08}

Thus, a reduction in wall thickness by 50% may lead to a reduction in the areal moments of inertia by approximately one half and may reduce the stiffness of the cross section to approximately 15%. Moreover, the wall thickness of the hollow member 2 may decrease continuously from the rear end 6 to the front end 5. In one embodiment, the wall thickness in the front third of the hollow member 2 may be, for example, at least about 20% less than the wall thickness in the rear third of the hollow member 2.

Furthermore, the porosity, as the ratio of the sum S of the n perforation areas to the total circumferential area M, may increase from the rear end 6 to the front end 5. For example, the porosity in the front third of the hollow member 2 may be at least about 20% greater than the porosity in the rear third of the hollow member 2. It should be noted that the smallest diameter of the n perforations 3 in the wall 4 of the hollow member 2 may be, for example, at least about 0.5 mm. In one embodiment, the maximum porosity attainable for the tightest possible arrangement of circular perforations 3 of the same size may be about 90%. However, in order to ensure the structural integrity of the wall 4 of the hollow member 2, the porosity should not exceed about 85%.

In order to increase the porosity (decrease stiffness) of the bone fixation element 1, the number of perforations 3 and/or the area/size of the perforations 3 may be increased. For example, the number of perforations 3 per tenth of the height of the hollow member 2 may increase in the direction of the front end 5, while the area of the individual perforations 3 may remain constant (i.e., the frequency or density of the perforations 3 may increase from the rear end 6 to the front end 5). Alternatively, the number of perforations 3 per tenth of the height of the hollow member 2 may remain constant in the direction of the front end 5, while the area of the individual perforations 3 may increase in the direction of the front end 5. Further, the partial view of the wall 4 of the hollow member 2, shown in FIG. 2, illustrates how the area/size of the perforations 3 may increase continuously from the rear end 6 to the front end 5. In such an embodiment, the stiffness of the hollow member 2 may correspondingly decrease along the longitudinal axis 7 from the rear end 6 to the front end 5.

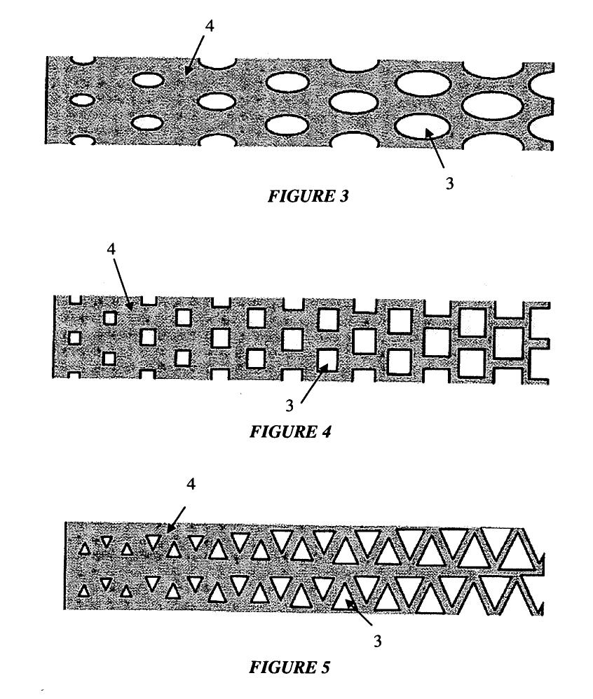

FIGS. 3 to 5 also illustrate various ways to change the porosity by changing the geometry of the perforations 3. Instead of round/circular perforations 3 (FIG. 2), the perforations 3 may be any other shape including, for example, oval (FIG. 3), square (FIG. 4) or angular (e.g., triangular or quadrilateral) (FIG. 5). These perforations 3 may also increase regularly in size from the rear end 6 to the front end 5. One purpose of the hole geometries may be to allow the stiffness of the bone fixation element 1 to decrease from the rear end 6 to the front end 5.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A device for insertion in bone comprising a hollow member having a longitudinal axis, a wall, a plurality of perforations in the wall, a front end and a rear end, wherein the hollow member has a stiffness which decreases along the longitudinal axis from the rear end to the front end.

2. The device of claim 1, wherein the stiffness of the hollow member decreases continuously from the rear end to the front end.

3. The device of claim 1, wherein the stiffness of the rear third of the hollow member is at least about 20% greater than the stiffness of the front third of the hollow member.

4. The device of claim 1, wherein the rear end is provided with means for accommodating a bone plate.

5. The device of claim 1, wherein the hollow member has a porosity which increases from the rear end of the hollow member to the front end of the hollow member.

6. The device of claim 5, wherein the porosity of the front third of the hollow member is at least about 20% greater than the porosity of the rear third of the hollow member.

7. The device of claim 1, wherein the hollow member has a wall thickness which decreases from the rear end of the hollow member to the front end of the hollow member.

8. The device of claim 7, wherein the wall thickness decreases continuously from the rear end of the hollow member to the front end of the hollow member.

9. The device of claim 7, wherein the wall thickness of the front third of the hollow member is at least about 20% less than the wall thickness of the rear third of the hollow member.

10. The device of claim 1, wherein each of the plurality of perforations has a diameter which is at least about 0.5 mm.

11. The device of claim 1, wherein the wall is a circumferential surface.

12. The device of claim 11 further comprising an external thread on at least a portion of the circumferential surface.

13. The device of claim 1, wherein each of the plurality of perforations has a shape selected from the group consisting of round, oval, square, triangular and quadrilateral.

14. The device of claim 1, wherein the front end is open.

15. A device for insertion in bone comprising a hollow cylinder having a longitudinal axis, a circumferential surface, a plurality of perforations in the circumferential surface, a front end and a rear end, wherein the hollow cylinder has a first stiffness at the front end and a second stiffness at the rear end, the first stiffness being less than second stiffness.

16. The device of claim 15, wherein the hollow cylinder has a height along the longitudinal axis and each of the plurality of perforations has an area, wherein the number of perforations per tenth of the height of the hollow cylinder increases in a first direction while the area of each of the plurality of perforations remains constant, wherein the first direction extends from the rear end to the front end.

17. The device of claim 15, wherein the hollow cylinder has a height along the longitudinal axis and each of the plurality of perforations has an area, wherein the number of perforations per tenth of the height of the hollow cylinder remains constant while the area of each of the plurality of perforations increases in the first direction, wherein the first direction extends from the rear end to the front end.

18. The device of claim 15, wherein the rear end is provided with means for accommodating a bone plate.

19. The device of claim 15, wherein the hollow cylinder has a first porosity at the front end and a second porosity at the rear end, the first porosity being greater than the second porosity.

20. The device of claim 19, wherein the hollow cylinder has a first thickness at the front end and a second thickness at the rear end, the first thickness being less than the second thickness.

21. The device of claim 15, wherein each of the plurality of perforations has a shape selected from the group consisting of round, oval, square, triangular and quadrilateral.

22. The device of claim 15, wherein the front end is open.
